SEQUENCE LISTING (3 sequences)

SEQ ID NO: 1 - DNA, 842 bp, Saccharomyces cerevisiae
  source 1..842: mol_type = genomic DNA
tccgtaggtgaacctgcggaaggatcattaaagaaatttaataattttgaaaatggattttttttgttttggcaagagcatgagagcttttactgggcaagaagacaagagatggagagtccagccgggcctgcgcttaagtgcgcggtcttgctaggcttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggtaacaaacacaaacaattttatttattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgtttttttttccaaagagaggtttctctgcgtgcttgaggtataatgcaagtacggtcgttttaggttttaccaactgcggctaatcttttttatactgagcgtattggaacgttatcgataagaagagagcgtctaggcgaacaatgttcttaaagtttgacctcaaatcaggtaggagtacccgctgaacttaagcatatcaataagcggagga

SEQ ID NO: 2 - DNA, 848 bp, Saccharomyces cerevisiae
  source 1..848: mol_type = genomic DNA
tccgtaggtgaacctgcggaaggatcattaaagaaatttaataattttgaaaatggattttttttttttgttttggcaagagcatgagagcttttactgggcaagaagacaagagatggagagtccagccgggcctgcgcttaagtgcgcggtcttgctaggcttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggttaacaaacacaaacaattttatctattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgttttttttttccaaagagaggtttctctgcgtgcttgaggtataatgcaagtacggtcgttttaggttttaccaactgcggctaatcttttttatactgagcgtattggaacgttatcgataagaagagagcgtctaggcgaacaatgttcttaaagtttgacctcaaatcaggtaggagtacccgctgaacttaagcatatcaataagcggagga

SEQ ID NO: 3 - DNA, 848 bp, Saccharomyces cerevisiae; identical to SEQ ID NO: 2
  source 1..848: mol_type = genomic DNA

(19) United States
(12) Patent Application Publication
(10) Pub. No.: US 2026/0226517 A1
(43) Pub. Date: Aug. 6, 2026

(54) YEAST MUTANT STRAIN HS-Y007 PRODUCING HIGH CONCENTRATION OF HEME, AND METHOD FOR HIGH PRODUCTION OF HEME USING SAME

(71) Applicant: HANWHA SOLUTIONS CORPORATION, Seoul (KR)

(72) Inventors: Seongjoon KANG, Seoul (KR); Jin-Hee PARK, Seoul (KR)

(73) Assignee: HANWHA SOLUTIONS CORPORATION, Seoul (KR)

(21) Appl. No.: 19/152,698

(22) PCT Filed: Jan. 31, 2024
(86) PCT No.: PCT/KR2024/001474

§ 371 Date: Jul. 31, 2025

(30) Foreign Application Priority Data
May 9, 2023 (KR) 10-2023-0060150

(51) Int. Cl. C12P 9/00; A23L 33/145; C12N 1/18; C12N 1/38; C12N 13/00; C12Q 1/689; C12R 1/865

(52) U.S. Cl. CPC C12P 9/00; A23L 33/145; C12N 1/18; C12N 1/38; C12N 13/00; C12Q 1/689; C12Q 2600/156; C12R 2001/865

(57) ABSTRACT

The present invention relates to a yeast mutant strain HS-Y007 producing a high concentration of heme, and a method for high production of heme using same. More specifically, provided are: a Saccharomyces yeast mutant strain which is prepared by emitting ultraviolet rays at a wild-type Saccharomyces cerevisiae in a specific condition, and which has better heme productivity than the wild-type; a method for preparing the yeast mutant strain; a method for producing heme by using the yeast mutant strain; a medium composition for culturing the yeast mutant strain; and a food composition comprising heme produced from the yeast mutant strain.

### TECHNICAL FIELD

The present invention relates to an HS-Y007 yeast mutant strain producing a high concentration of heme and a method for producing heme at high concentrations using the same, and more specifically to a Saccharomyces yeast mutant strain having enhanced heme productivity compared to a wild type, which is produced by irradiating wild-type Saccharomyces cerevisiae with ultraviolet rays under specific conditions, a method for producing the yeast mutant strain, a method for producing heme using the yeast mutant strain, a medium composition for culturing the yeast mutant strain, and a food composition including heme that is produced from the yeast mutant strain.

### BACKGROUND ART

Interest in alternative protein materials is increasing as a solution to problems such as global warming, food shortage and animal welfare. In addition, as the number of vegetarians increases globally along with consumer interest in health, interest in alternative meat is also increasing. In line with this social trend, the development and launch of various alternative meat products is actively taking place, and the market for plant-based meat substitutes that can nutritionally replace animal meat is continuously growing.

The early plant-based alternative meat manufacturing technology began with textured vegetable protein (TVP) as the main raw material, and it has been developed in the following order of diversifying raw materials and studying the properties of each raw material, implementing the texture of meat using high-moisture molding technology, and producing and adding heme, which is a key element of meat flavor, using biotechnology.

Impossible Foods, which is a food tech company that is considered the most technologically advanced in the alternative meat market, produces and adds heme, which plays an important role in adding flavor to meat, using genetically modified organism (GMO) yeast. Related technology is disclosed in Patent Document 1. However, consumers who oppose GMO-derived foods have a negative perception of the consumption of heme produced using GMO yeast. Meanwhile, in Korea, as in the United States and Europe, interest in alternative meat is increasing, and the consumption of related foods is also increasing. However, currently produced alternative meat relies on imports for most of its main raw materials, and thus, the situation is that research on replacing the same with domestic resources is needed. In particular, as regulations on GMO-derived foods are being strengthened in Korea, there are restrictions on adding heme produced by using GMO yeast to alternative meat.

Meanwhile, heme is generally extracted from animal blood using organic solvents or enzymatic hydrolysis, but the safety of heme separated and purified from slaughter blood is problematic due to the risk of diseases that can be caused in animals, such as mad cow disease. Since the content in the blood is low, a lot of blood is required, and because animal slaughter is required when using this method, vegetarian consumers do not prefer to consume alternative meat containing heme extracted from animal slaughter.

Saccharomyces cerevisiae is a single-celled eukaryote and is a Generally Recognized as Safe (GRAS) strain that has been used for alcohol and baking purposes in food processing for a long period of time. In the present invention, the inventors of the present invention sought to develop a technology that can enhance the heme productivity of yeast in a simpler method using wild-type yeast. Accordingly, among various yeast strains already used in the food industry, wild-type yeast with high heme productivity was selected as a parent strain, and the parent strain was irradiated with ultraviolet rays under specific conditions to produce a mutant strain with enhanced heme productivity compared to a wild type, thereby completing the present invention.

### Related Art Documents

### Patent Documents

(Patent Document 1) Korean Registered Patent No. 10-2229968

### DISCLOSURE

### Technical Problem

An object of the present invention is to provide a Saccharomyces yeast mutant strain with enhanced heme productivity compared to a wild type, which is produced by irradiating wild-type Saccharomyces cerevisiae with ultraviolet rays under specific conditions.

Another object of the present invention is to provide a method for producing a Saccharomyces yeast mutant strain with enhanced heme productivity compared to a wild type, a method for producing heme using the yeast mutant strain, and a medium composition for culturing the yeast mutant strain.

Still another object of the present invention is to provide a food composition including heme that is produced from the yeast mutant strain.

### Technical Solution

In order to solve the above-described problems, the present invention provides an HS-Y007 mutant strain of Saccharomyces cerevisiae with enhanced heme productivity compared to a wild type (Accession No. KCTC 15340BP).

In this case, the HS-Y007 mutant strain may be derived from Saccharomyces cerevisiae KCCM12638.

In this case, the HS-Y007 mutant strain may be produced by irradiating Saccharomyces cerevisiae KCCM12638 with ultraviolet rays having a wavelength of 250 to 260 nm at a distance of 20 to 50 cm in the vertical direction at an irradiation dose of 3,000 to 9,000 mW/cm² for 1 to 2 hours.

In this case, the HS-Y007 mutant strain may have single nucleotide sequence mutations of 413A>T and 468T>A mutations in chromosome 3, a 570166G>T mutation in chromosome 5, 8579T>C, 158964T>A and 171975G>C mutations in chromosome 7, a 364680A>T mutation in chromosome 8, a 502139A>C mutation in chromosome 10, 1064659G>C, 1064798T>G and 1064817G>T mutations in chromosome 12, a 783791C>T mutation in chromosome 14, and a 30149A>T mutation in chromosome 15, compared to Saccharomyces cerevisiae KCCM12638.

In addition, the present invention provides a method for producing an HS-Y007 mutant strain of Saccharomyces cerevisiae with enhanced heme productivity compared to a wild type (Accession No. 15340BP), including:
a) pre-culturing Saccharomyces cerevisiae KCCM12638; and
b) irradiating the pre-cultured Saccharomyces cerevisiae KCCM12638 with ultraviolet rays.

In this case, the method may further include a′) diluting the pre-cultured strain such that the optical density at 600 nm is 0.5 to 1.5, between step a) and step b).

In this case, step b) may be performed by irradiating Saccharomyces cerevisiae KCCM12638 with ultraviolet rays having a wavelength of 250 to 260 nm at a distance of 20 to 50 cm in the vertical direction at an irradiation dose of 3,000 to 9,000 mW/cm² for 1 to 2 hours.

In this case, the method may further include c) selecting a mutant strain that has single nucleotide sequence mutations of 413A>T and 468T>A mutations in chromosome 3, a 570166G>T mutation in chromosome 5, 8579T>C, 158964T>A and 171975G>C mutations in chromosome 7, a 364680A>T mutation in chromosome 8, a 502139A>C mutation in chromosome 10, 1064659G>C, 1064798T>G and 1064817G>T mutations in chromosome 12, a 783791C>T mutation in chromosome 14, and a 30149A>T mutation in chromosome 15, compared to Saccharomyces cerevisiae KCCM12638, after step b).

In addition, the present invention provides a method for producing heme using the HS-Y007 mutant strain of Saccharomyces cerevisiae (Accession No. KCTC 15340BP) described above.

In this case, the method may include the following steps:
a) culturing an HS-Y007 mutant strain of Saccharomyces cerevisiae (Accession No. KCTC 15340BP); and
b) recovering heme from the HS-Y007 mutant strain.

In this case, the HS-Y007 mutant strain of step a) may be cultured in a medium including at least one selected from the group consisting of glucose, fructose or galactose as a carbon source, and yeast extract, peptone, tryptone, malt extract, beef extract, casein, soytone, sodium nitrate (NaNO₃), ammonium chloride (NH₄Cl) and ammonium sulphate ((NH₄)₂SO₄) as a nitrogen source.

In this case, the nitrogen source may be 2 to 6% (w/v) of yeast extract and 0.1 to 3% (w/v) of peptone.

In this case, the HS-Y007 mutant strain of step a) may be cultured in a fed-batch manner at a pH of 3 to 6, with a shaking speed of 100 to 1,500 rpm, and air injection at a speed of 0.5 to 10 vvm.

Additionally, the present invention provides a medium composition for culturing an HS-Y007 mutant strain of Saccharomyces cerevisiae (Accession No. KCTC 15340BP), including:
i) 3 to 7% (w/v) of glucose, fructose or galactose as a carbon source; and
ii) 2 to 6% (w/v) of yeast extract and 0.1 to 3% (w/v) of peptone as a nitrogen source.

In this case, the medium composition may be a medium for producing heme.

Furthermore, the present invention provides a food composition, including an HS-Y007 mutant strain of Saccharomyces cerevisiae with enhanced heme productivity compared to a wild type (Accession No. KCTC 15340BP), a culture product of the HS-Y007 mutant strain, heme produced from the HS-Y007 mutant strain or heme isolated from a culture product of the HS-Y007 mutant strain.

In this case, the food may be artificial meat or meat substitute food.

In addition, the present invention provides the use of the medium composition described above for increasing heme production of an HS-Y007 mutant strain of Saccharomyces cerevisiae (Accession No. KCTC 15340BP).

### Advantageous Effects

The HS-Y007 mutant strain of Saccharomyces cerevisiae (Accession No. KCTC 15340BP) according to the present invention is produced by irradiating ultraviolet rays under specific conditions to wild-type Saccharomyces cerevisiae KCCM12638, which is already used in the food industry, and thus, it is possible to secure a yeast strain exhibiting high heme productivity in a much simpler and simpler manner than existing GMO yeast. The HS-Y007 mutant strain of Saccharomyces cerevisiae (Accession No. KCTC 15340BP) produced in this manner exhibits enhanced heme productivity compared to the wild type, thereby enabling more efficient heme production.

### DESCRIPTION OF DRAWINGS

FIG. 1 is a mimetic diagram showing a method for extracting heme that is present in yeast strain cells.

FIG. 2 shows the results of fed-batch culture of Saccharomyces cerevisiae KCCM12638 strain using 40YP20D medium (4% (w/v) yeast extract (40 g/L), 2% (w/v) bacto peptone (20 g/L), 2% (w/v) glucose (20 g/L)) at a pH 3.5 condition. (A) shows the cell, metabolite and heme concentrations, (B) shows the dissolved oxygen (DO %), and (C) shows the feeding solution injection rate.

FIG. 3 shows the results of fed-batch culture of Saccharomyces cerevisiae KCCM12638 strain using 40YP20D medium under pH 4.5 conditions. (A) shows the cell, metabolite and heme concentrations, (B) shows the dissolved oxygen (DO %), and (C) shows the feeding solution injection rate.

FIG. 4 is a graph showing the survival rate of Saccharomyces cerevisiae KCCM12638 strain according to ultraviolet irradiation time.

FIG. 5 shows the results of comparison by performing BLAST for the ITS (internal transcribed spacer) portions (ITS1, 5.8S rRNA and ITS2 base sequences) of Saccharomyces cerevisiae S288C and Saccharomyces cerevisiae KCCM12638, which is the parent strain of the mutant yeast of the present invention. In this case, the query is the ITS portion (ITS1, 5.8S rRNA, ITS2) of Saccharomyces cerevisiae S288C, and the subject is the ITS portion (ITS1, 5.8S rRNA, ITS2) of Saccharomyces cerevisiae KCCM12638. The query sequence is shown in SEQ ID NO: 1, and the subject sequence is shown in SEQ ID NO: 2.

FIG. 6 shows the results of comparison by performing BLAST for the ITS portions (ITS1, 5.8S rRNA and ITS2 base sequences) of the parent strain Saccharomyces cerevisiae KCCM12638 and an HS-Y007 mutant strain of Saccharomyces cerevisiae derived therefrom (Accession No. KCTC 15340BP). In this case, the query is the ITS portion (ITS1, 5.8S rRNA, ITS2) of Saccharomyces cerevisiae KCCM12638, and the subject is the ITS portion (ITS1, 5.8S rRNA, ITS2) of Saccharomyces cerevisiae HS-Y007 (Accession No. KCTC 15340BP). The query sequence is shown in SEQ ID NO: 2, and the subject sequence is shown in SEQ ID NO: 3.

### BEST MODE

Hereinafter, the present invention will be described in more detail.

All technical terms used in the present invention, unless otherwise defined, are used with the same meanings as commonly understood by a person skilled in the art in the field related to the present invention. In addition, preferred methods and samples are described in the present specification, but similar or equivalent methods are also included in the scope of the present invention.

All numbers expressing the size, quantity and physical properties of features used in the present specification and claims are to be understood in all instances as being modified by the term “about.” Accordingly, unless indicated to the contrary, the numerical parameters disclosed in the present specification and claims are approximations that may vary depending on the desired properties sought to be achieved by a person skilled in the art using the teachings disclosed herein.

In response to the need to develop a technology that can improve the heme productivity of yeast in a simpler way using wild-type yeast, the inventors of the present invention selected a wild-type yeast having a high hem output among various yeast strains already used in the food industry, and irradiated ultraviolet rays of certain conditions to a parent strain to prepare mutant strains with enhanced hem productivity compared to a wild type.

Therefore, a first aspect of the present invention relates to an HS-Y007 mutant strain of Saccharomyces cerevisiae (Accession No. KCTC 15340BP) with enhanced heme productivity compared to a wild type, and a method for producing the same.

In a specific embodiment of the present invention, in order to produce a yeast strain with a high heme content that can be directly applied to the food industry, a yeast strain with a high heme content was first selected among various yeast strains already used in the food industry and used as a parent strain. According to the selection results, it was confirmed that Saccharomyces cerevisiae KCCM12638, which is a type of wild-type Saccharomyces cerevisiae (S. cerevisiae), had a higher heme productivity than other yeast strains, and was selected as the parent strain.

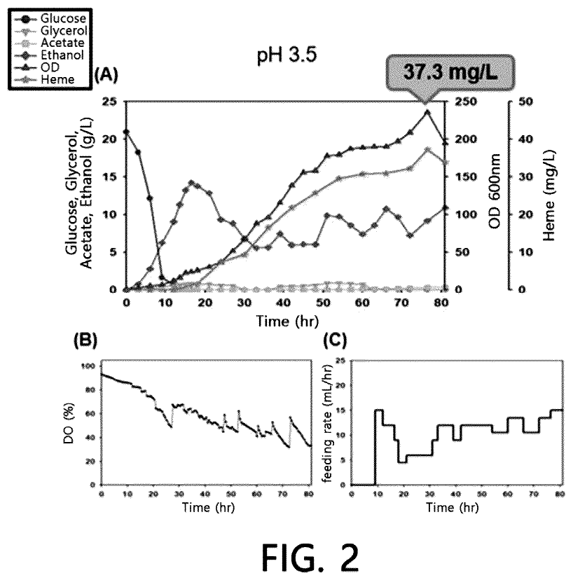

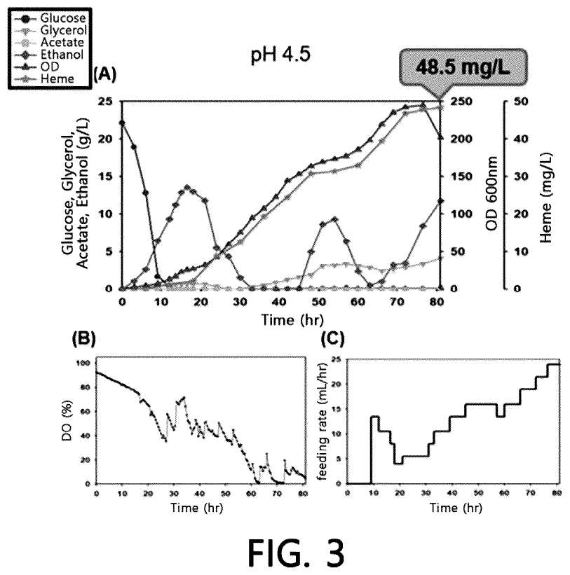

In another specific embodiment of the present invention, in order to optimize the culture conditions of S. Cerevisiae KCCM12638, which was selected as a parent strain, the parent strain was inoculated in 40YP20D medium including glucose as a carbon source and yeast extract and bacto peptone as nitrogen sources, and fed-batch culture was performed by adjusting the acidity, shaking speed and air injection speed of the culture medium. As a result, as shown in FIGS. 2 and 3, it was confirmed that the parent strain exhibited the highest hem productivity when it was fed-batch cultured by injecting air at a speed of 0.5 to 10 vvm at pH 3 to 6 at a shaking speed of 100 to 1,500 rpm.

In this case, the glucose included in the medium may be replaced with another substance commonly used as a carbon source, for example, fructose or galactose, but is not limited thereto. In addition, as nitrogen sources included in the medium, the yeast extract may be used at 2 to 6% (w/v) and peptone may be used at 0.1 to 3% (w/v), but is not limited thereto. A person skilled in the art can appropriately select the type and concentration of a carbon source and nitrogen source included in the medium according to the culture conditions.

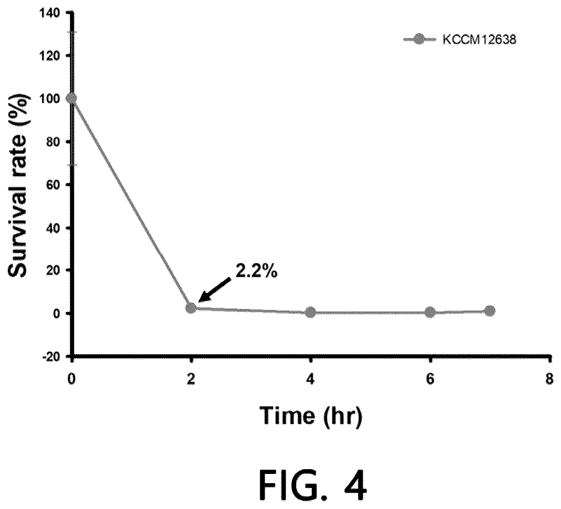

In a specific embodiment of the present invention, UV rays were irradiated to S. Cerevisiae KCCM12638, which exhibited high hem productivity, to produce a mutant yeast strain with enhanced hem productivity compared to a wild type. For the production of a mutant yeast strain, a PCR tube containing the parent strain was placed for 2 to 12 hours at a 30 cm distance below the 254 nm UV lamp. As a result, as shown in FIG. 4, it was confirmed to show a death rate of 97.8% at 2 hours after the UV irradiation, and this time was selected as the optimal UV irradiation time.

As a result of randomly selecting the colonies that emerged after UV treatment and analyzing the heme concentration, a mutant yeast strain with an increased heme content of about 21% compared to the parent strain was discovered, which was named Saccharomyces cerevisiae HS-Y007. It was deposited at the Korea Research Institute of Bioscience and Biotechnology Korean Collection for Type Cultures Gene Bank (KCTC) on Mar. 7, 2023, and was assigned the accession number KCTC 15340BP.

Therefore, the HS-Y007 mutant strain of Saccharomyces cerevisiae (Accession No. KCTC 15340BP) with enhanced heme productivity compared to a wild type may be derived from Saccharomyces cerevisiae KCCM12638, and it may be produced by irradiating Saccharomyces cerevisiae KCCM12638 with ultraviolet rays having a wavelength of 250 to 260 nm at a dose of 3,000 to 9,000 mW/cm² at a distance of 20 to 50 cm in the vertical direction for 1 to 2 hours.

In this case, if the distance from which ultraviolet rays are irradiated is less than 20 cm in the vertical direction, a problem may arise where the mortality rate increases significantly even with a short period of ultraviolet irradiation, and if the distance from which ultraviolet rays are irradiated exceeds 50 cm in the vertical direction, there may be problems in that mutations are not induced even in a long period of ultraviolet irradiation.

In this case, if the UV irradiation time is less than 1 hour, it may be difficult to select strains with induced mutations due to the low death rate. Since there is little change in the death rate over time after 2 hours of irradiating ultraviolet rays, it is unnecessary to irradiate ultraviolet rays for more than 2 hours.

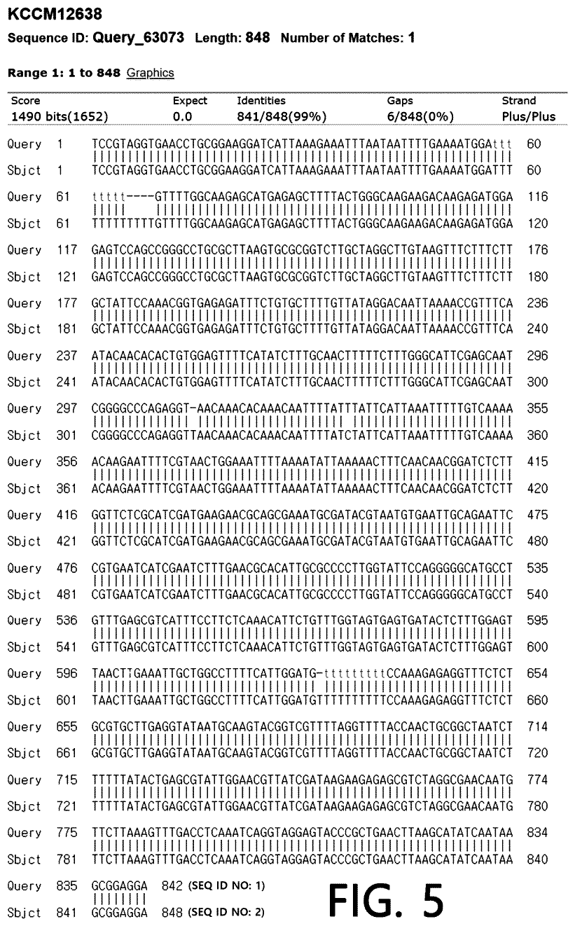

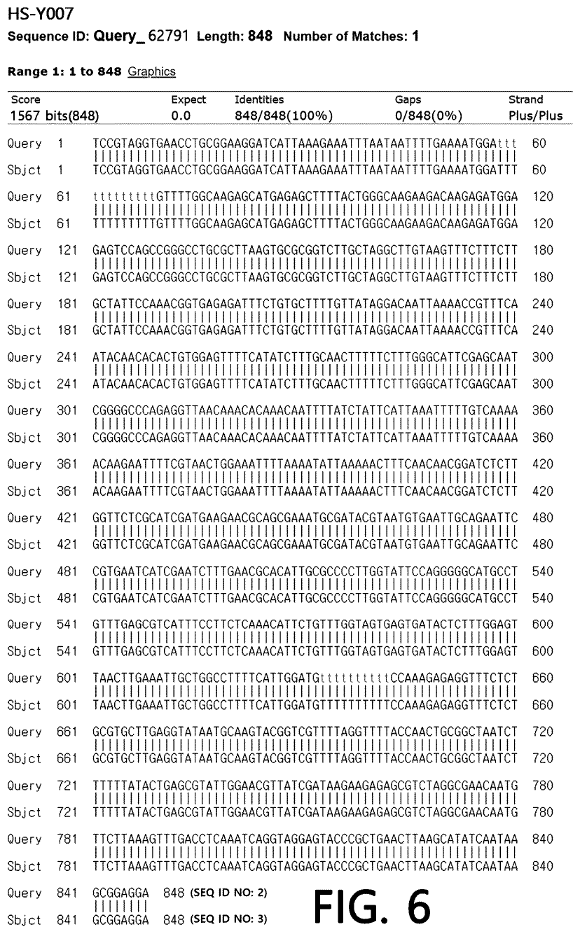

The inventors of the present invention used whole genome re-sequencing to determine the distribution and pattern of mutations at the genome level of an HS-Y007 mutant strain of Saccharomyces cerevisiae (Accession No. KCTC 15340BP) with enhanced heme productivity compared to a wild type. For this purpose, first of all, Saccharomyces cerevisiae S288C (S. cerevisiae S288C) was used as the full-length genome to secure single nucleotide polymorphism (SNP) information between the parent strain, KCCM12638 and the UV mutant strain HS-Y007 (Accession No. KCTC 15340BP), and ITS1, 5.8S rRNA and ITS2 base sequences (hereinafter, referred to as ‘ITS portion’) were extracted from the base sequences obtained through whole-genome resequencing, and BLAST was performed to compare the ITS portions of UV mutant strain HS-Y007 (Accession No. KCTC 15340BP) and the parent strain, KCCM12638. As a result, as shown in FIG. 5, 841 base sequences out of 848 KCCM12638 ITS portions matched S288C, and as shown in FIG. 6, 848 out of 848 base sequences of the ITS portions of KCCM12638 and the UV mutant strain HS-Y007 (Accession No. KCTC 15340BP) all matched, which confirmed that the UV mutant strain HS-Y007 is also a Saccharomyces cerevisiae strain derived from KCCM12638.

Next, in order to determine whether the UV mutant strain HS-Y007 (Accession No. KCTC 15340BP) was an independently produced strain modified from KCCM12638, the SNPs of the KCCM12638 and HS-Y007 (Accession No. KCTC 15340BP) were compared. As a result, as shown in Table 4, it was confirmed that HS-Y007 (Accession No. KCTC 15340BP) had at least 13 SNPs compared to the KCCM12638 strain, that is, SNPs of 413A>T and 468T>A mutations in chromosome 3, a 570166G>T mutation in chromosome 5, 8579T>C, 158964T>A and 171975G>C mutations in chromosome 7, a 364680A>T mutation in chromosome 8, a 502139A>C mutation in chromosome 10, 1064659G>C, 1064798T>G and 1064817G>T mutations in chromosome 12, a 783791C>T mutation in chromosome 14, and a 30149A>T mutation in chromosome 15.

The HS-Y007 mutant strain (Accession No. KCTC 15340BP) of Saccharomyces cerevisiae produced in this way shows enhanced heme productivity compared to the parent strain, wild-type Saccharomyces cerevisiae KCCM12638, and thus, this can be used in a method for industrially mass-producing heme.

Therefore, a second aspect of the present invention relates to a method for producing heme using the above-described HS-Y007 mutant strain of Saccharomyces cerevisiae (Accession No. KCTC 15340BP) and a medium composition for culturing the strain.

Specifically, the method for producing heme may include a) culturing an HS-Y007 mutant strain of Saccharomyces cerevisiae (Accession No. KCTC 15340BP); and b) recovering heme from the HS-Y007 mutant strain.

In the present invention, the culturing in step a) may be performed by using the pre-culture medium in which the strain was cultured, by using an isolated strain, or by using a frozen strain or a culture product thereof.

As used herein, the term “culture” means growing the strain under appropriately artificially controlled environmental conditions. The culture method according to the present invention includes batch culture, continuous culture and fed-batch culture, and specifically, it may be cultured continuously in a batch process, a fed batch or repeated fed batch process, but is not limited thereto.

The culture according to the present invention is not particularly limited, but may be cultured by using, for example, a liquid culture tank, a rotary drum fermentor or a tray fermentor. In addition to the rotary drum type or tray fermenter, if it is useful for the fermentation of strains, it may be used in the method of the present invention without restrictions on its type, and an appropriate device may be selected and used depending on the production scale.

In the present invention, the pH of the culture may be adjusted by adding compounds such as ammonium hydroxide, potassium hydroxide, ammonia, phosphoric acid and sulfuric acid to the culture in an appropriate manner. During culturing, foam generation may be suppressed by using an antifoaming agent such as fatty acid polyglycol ester. Additionally, in order to maintain the aerobic state of the culture, oxygen or oxygen-containing gas may be injected into the culture.

According to a specific embodiment of the present invention, the HS-Y007 mutant strain of Saccharomyces cerevisiae (Accession No. KCTC 15340BP) of step a) above may be cultured in the same medium as the medium in which its parent strain, Saccharomyces cerevisiae KCCM12638, is cultured.

For example, the strain of step a) above may be cultured in a medium including at least one selected from the group consisting of glucose, fructose or galactose as a carbon source, and yeast extract, peptone, tryptone, malt extract, beef extract, casein, soytone, sodium nitrate (NaNO₃), ammonium chloride (NH₄Cl) and ammonium sulphate ((NH₄)₂SO₄) as a nitrogen source. Specifically, the strain of step a) may be cultured in a medium including 3 to 7% (w/v) of glucose, fructose or galactose as a carbon source, 2 to 6% (w/v) of yeast extract and 0.1 to 3% (w/v) of peptone as a nitrogen source, but is not limited thereto, and a person skilled in the art may appropriately select the types and concentrations of carbon sources and nitrogen sources included in the medium according to culture conditions for the purpose of increasing heme productivity.

In addition, the culture conditions of the HS-Y007 mutant strain of Saccharomyces cerevisiae (Accession No. KCTC 15340BP) in step a) may be cultured in the same conditions for culturing its parent strain, Saccharomyces cerevisiae KCCM12638.

For example, the strain in step a) may be cultured in a fed-batch manner at pH 3 to 6, and more preferably, pH 3 to 5.5, with a shaking speed of 100 to 1,500 rpm and air injection at a rate of 0.5 to 10 vvm, but is not limited thereto, and a person skilled in the art may appropriately adjust pH, shaking speed, air injection speed and the like for the purpose of increasing heme productivity.

In this case, an additional feed solution may be injected at the time when all of the initial glucose is consumed in step a). The feed solution may include various carbon sources and/or various nitrogen sources like the initial medium, and may include the same or different carbon and/or nitrogen sources as the initial medium. In a specific embodiment of the present invention, a feed solution including the same initial medium but high concentrations of carbon and nitrogen sources was injected, but a person skilled in the art may appropriately select and control the components and concentrations included in the feed solution by considering the components and culture conditions of the initial medium.

The injection rate of the feed solution may be determined within a range where the glucose concentration of the culture medium is close to 0 g/L and the ethanol concentration does not exceed 10 g/L. The injection rate of the feed solution including the carbon source and the nitrogen source within the above range may be, for example, 1 to 50 mL/hr, preferably 2 to 35 mL/hr, but is not limited thereto.

In the method for producing heme according to the present invention, step b) may be performed by using a method known in the art for recovering heme by disrupting a yeast strain.

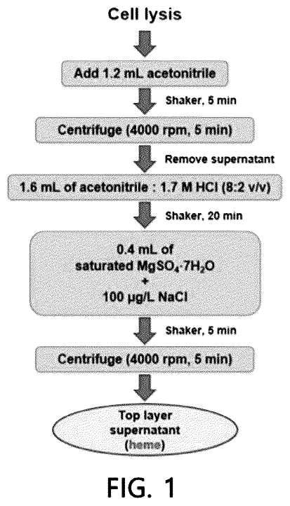

According to a specific embodiment of the present invention, step b) may be performed by a series of processes shown in FIG. 1.

The process of FIG. 1 is briefly described as follows.

First of all, the yeast strain that has undergone the culture process in step a) is centrifuged, and then, 0.4 mL of yeast lysis buffer (e.g. Y-PER™ Yeast Protein Extraction Reagent) per OD₆₀₀*mL=60 is added to disrupt the yeast cells, and by adding 1.2 mL of acetonitrile (ACN) to 0.4 mL of crushed yeast extract, the reaction is performed for 5 minutes, and it is centrifuged to remove the supernatant, and collect only the precipitate. To the recovered precipitate, 1.6 mL of a 1.7 M hydrochloric acid (HCl) solution of acetonitrile prepared by mixing in a volume ratio of 8:2 is added, and the mixture is reacted for 20 minutes. In addition, 0.4 mL of a solution including saturated concentrations of MgSO₄ and 100 μg/L NaCl at room temperature is added, and the mixture is shaken for 5 minutes. Centrifugation is performed to separate the aqueous layer and the acetonitrile layer, and the acetonitrile layer including heme is recovered.

Methods for recovering heme in the present invention may include centrifugation, filtration, anion exchange chromatography, crystallization and HPLC, but are not limited thereto.

In the method for producing heme according to the present invention, in step b), heme may be recovered by harvesting the strain before the heme concentration in the medium reaches 0.5 mM. This is because when the heme concentration in the medium reaches 0.5 mM, yeast growth may be inhibited, and heme production may decrease.

In the present invention, step b) may include additional recovery of heme from the strain culture medium.

In the method for producing heme according to the present invention, the culture medium used in step a) may be optimized to increase heme productivity of the HS-Y007 mutant strain of Saccharomyces cerevisiae (Accession No. KCTC 15340BP).

Accordingly, a second aspect of the present invention provides a medium composition for culturing an HS-Y007 mutant strain of Saccharomyces cerevisiae (Accession No. KCTC 15340BP).

The medium composition according to the present invention may include the medium used in step a) of the above-described method for producing heme, and for example, it may include i) 3 to 7% (w/v) of glucose, fructose or galactose as a carbon source; and ii) 2 to 6% (w/v) yeast extract and 0.1 to 3% (w/v) peptone as a nitrogen source, but is not limited thereto.

In the present invention, when the above medium composition is used, the HS-Y007 mutant strain of Saccharomyces cerevisiae (Accession No. KCTC 15340BP) shows enhanced heme productivity compared to its parent strain, Saccharomyces cerevisiae KCCM12638, and thus, the medium composition may be used as a medium composition for producing heme.

Accordingly, the second aspect of the present invention provides the use of the above-described composition for increasing heme production in an HS-Y007 mutant strain of Saccharomyces cerevisiae (Accession No. KCTC 15340BP), wherein the composition may include i) 3 to 7% (w/v) glucose, fructose or galactose as carbon source; and ii) 2 to 6% (w/v) yeast extract and 0.1 to 3% (w/v) peptone as a nitrogen source.

Furthermore, the second aspect of the present invention provides the use of the above-described composition for the production of a culture medium for increasing heme production of an HS-Y007 mutant strain of Saccharomyces cerevisiae (Accession No. KCTC 15340BP), wherein the composition may include i) 3 to 7% (w/v) of glucose, fructose or galactose as a carbon source; and ii) 2 to 6% (w/v) yeast extract and 0.1 to 3% (w/v) peptone as a nitrogen source.

Heme produced as described above may be used in various foods.

Accordingly, a third aspect of the present invention relates to a food composition including an HS-Y007 mutant strain of Saccharomyces cerevisiae (Accession No. KCTC 15340BP), a culture product of the strain, heme produced from the strain or heme isolated from a culture product of the strain.

In the present invention, the strain may be one or more selected from the group consisting of the strain itself, a strain lysate, a strain concentrate, a strain extract and strain dry matter.

As used herein, the term “lysate” refers to something obtained by physically and chemically treating the strain itself, high-pressure sterilization, or ultrasonic treatment, or by crushing the supernatant, fermentation product or culture solution obtained by centrifuging the culture of the strain, but is not limited thereto.

As a term of the present invention, “fermented product” may include a strain obtained by culturing a microorganism in a medium for a certain period of time, a medium including its metabolites, extra nutrients and the like, a culture medium from which the strain is removed from the medium, and a dilution or concentrate of the above medium or culture medium, or a dried product thereof. The term “fermentation” means that it is not a putrefaction reaction during the process in which microorganisms decompose organic matter using their own enzymes.

In the present invention, the method of obtaining a fermented product from the strain is not particularly limited, and it may be obtained according to a method commonly used in the relevant technical field or similar fields.

In the present invention, the fermentation product obtained from the strain may include not only the fermented material itself, but also a culture medium of the strain in which the strain and the culture coexist, a fermentation product obtained by filtering the strain from the culture medium, a supernatant obtained by centrifuging the culture medium to remove the strain, a fermentation product obtained by sterilizing the strain from the culture medium and filtering the same, an extract obtained by extracting the fermentation product or the culture medium including the same, a diluted solution obtained by diluting the fermentation product or an extract thereof, a dried product obtained by drying the fermentation product or an extract thereof, a lysate obtained by capturing and crushing the cells of the strain, and all kinds of materials including the fermentation product generated from the strain.

As used herein, the term “extract” means a result such as a liquid component obtained by immersing a target substance in various solvents and then extracting the same for a certain period of time at room temperature, low temperature or heated conditions, or a solid component obtained by removing a solvent from the liquid component. In addition, it may be comprehensively interpreted to include a dilution of the result, a concentrate thereof, a controlled substance thereof, a purified substance and the like, in addition to the result.

As used herein, the term “dried material” refers to a product from which moisture has been removed from the strain itself or a culture, and it may include microbial cells and cultures dried by known drying methods such as spray drying, freeze drying and vacuum drying.

As used herein, the term “culture product” refers to a product obtained by culturing the above strain in a medium. For example, the culture product of the present invention may include a component remaining in a culture medium after harvesting the strain from the culture medium of the HS-Y007 mutant strain of Saccharomyces cerevisiae (Accession No. KCTC 15340BP), or may include a component of a culture medium including the strain. As used herein, the term “culture solution” means a portion of the culture excluding the strain. For example, the culture solution of the present invention may include a portion of the culture supernatant excluding the strain after centrifugation from the culture medium of the mutant strain HS-Y007 of Saccharomyces cerevisiae (Accession No. KCTC 15340BP), or a portion of the culture supernatant excluding the lysate.

The culture product may be an HS-Y007 mutant strain of Saccharomyces cerevisiae (Accession No. KCTC 15340BP), a total culture of the strain, a culture medium, a fraction thereof and the like. The culture medium may be a culture supernatant of the strain. In this case, the culture supernatant may be obtained by centrifuging the culture of the strain, and the fraction may be obtained by subjecting the strain, the culture, culture supernatant and the like to methods such as centrifugation and chromatography.

In the present invention, the food composition may include 0.02 to 80 wt %, and preferably, 0.02 to 50 wt %, of the strain, a culture product of the strain, heme produced from the strain, or heme isolated from the culture of the strain, based on the total weight of the composition, but is not limited thereto, and the content thereof may be appropriately changed depending on the type of food, the food consumption target, the consumption purpose and the like.

In the present invention, the food may be artificial meat or meat substitute food, but is not limited thereto. In the present invention, the artificial meat or meat substitute food refers to a meat replica that is imitated to have a texture, texture, flavor and/or flavor similar to meat.

The food composition is not particularly limited as long as it is an edible food. Non-limiting examples of the food include any animal-based or non-animal-based (e.g., plant-based) food product in the form of hot dogs, burgers, meat cuts, sausages, steaks, fillets, grilled meats, breasts, thighs, wings, meatballs, meatloaf, bacon, strips, fingers, nuggets, cutlets or cubes, or a combination of animal-based or non-animal-based food products.

In another aspect, the food may be provided in the form of a soup or stew base, a bouillon, and for example, a powder or cube, a flavor packet, or a food additive such as a seasoning packet or shaker.

### MODES OF THE INVENTION

Hereinafter, the present invention will be described in more detail through examples. However, the present invention can be modified in various ways and can have various forms, and the specific examples and descriptions described below are only intended to help understanding the present invention, and are not intended to limit the present invention to specific disclosed forms. It should be understood that the scope of the present invention includes all modifications, equivalents or substitutes included in the spirit and technical scope of the present invention.

### EXAMPLE 1

### Selection and Analysis of Yeast Parent Strain

### 1-1. Selection of Parent Strain

In order to produce a yeast strain with high heme content that can be directly applied to the food industry, S. cerevisiae KCCM12638, which is a type of wild-type Saccharomyces cerevisiae used in whiskey production, was acquired from the Korean Culture Center of Microorganisms. In order to determine the culture performance and heme content of the parent strain, the following processes of 1-2 to 1-5 were performed.

### 1-2. Preculture

50 μL of a yeast glycerol stock stored in a deep freezer was inoculated into test tubes including 5 mL YP20D (1% (w/v) yeast extract (10 g/L), 2% (w/v) bacto peptone (20 g/L), 2% (w/v) glucose (20 g/L)) medium and pre-cultured at 25° C. and 250 rpm for 48 hours.

### 1-3. Main Culture and Yeast Cell Disruption

The pre-cultured yeast strain was inoculated into a baffled flask including 100 mL of YP50D (1% (w/v) yeast extract (10 g/L), 2% (w/v) bacto peptone (20 g/L), 5% (w/v) glucose (50 g/L)) medium such that the optical density at 600 nm (OD600) became 1.0. The yeast strain cells, which were cultured under shaking conditions of 25° C. and 250 rpm for 48 hours, were collected by centrifugation until OD600XmL=60 (e.g., if OD600=1, 60 mL of culture medium), and then, 0.4 mL of Y-PER Yeast Protein Extraction Reagent (Thermo Fisher Scientific Inc., Rockford, IL, USA) was added to disrupt the yeast cells.

### 1-4. Heme Concentration Measurement

As shown in FIG. 1, the yeast cell disruption solution was pretreated to measure the heme concentration present in the yeast extract. According to the procedure in FIG. 1, 1.2 mL of acetonitrile (ACN) was added to 0.4 mL of yeast extract and reacted for 5 minutes and then centrifuged, the supernatant was removed, and only the precipitate was collected. To the recovered precipitate, 1.6 mL of acetonitrile: 1.7 M hydrochloric acid (HCl) solution prepared by mixing at a volume ratio of 8:2 was added and reacted for 20 minutes. Additionally, 0.4 mL of a solution including a saturated concentration of MgSO₄ and 100 μg/L NaCl at room temperature was added and shaken for 5 minutes, followed by centrifugation to separate the aqueous solution layer and the acetonitrile layer. The acetonitrile layer including heme was taken and subjected to HPLC analysis.

The concentration of heme was measured by using high performance liquid chromatography (HPLC) (Thermo fisher Ultimate 3000) equipped with a YMC-Pack ODS-A, 12 nm, 5 μm, 150×4.6 mm column (YMC, Japan) and UV detector. Detailed analysis conditions are shown in Tables 1 and 2. Hemin (Sigma, USA) was used as a heme standard material.

TABLE 1
HPLC Conditions

| Mobile phase A | ACN/H₂O (5:95 v/v) + 0.1% Formic acid | |
|---|---|---|
| Mobile phase B | ACN/H₂O (95:5 v/v) + 0.1% Formic acid | |
| Flow rate | 0.4 | mL/min |
| Injection volume | 20 | μL |
| Autosampler temperature | 5° | C. |
| Colum temperature | 50° | C. |

| | |
|---|---|
| Detector | UV-Vis at 400 nm |

TABLE 2

| | Time [min] | A (%) | B (%) |
|---|---|---|---|
| Gradient | 0 | 80 | 20 |
| | 10 | 0 | 100 |
| | 11 | 0 | 100 |
| | 11.1 | 0 | 100 |
| | 12 | 80 | 20 |
| | 25 | 80 | 20 |

Saccharomyces cerevisiae D452-2, which is a common laboratory yeast strain, was used as a control and compared with the heme concentration of Saccharomyces cerevisiae KCCM12638, which is a yeast strain for making American whiskey. Saccharomyces cerevisiae D452-2 produced 2.5 mg/L of heme, whereas Saccharomyces cerevisiae KCCM12638 produced 8.6 mg/L, and thus, it was found that the heme production was high compared to other yeast strains. Based on the results of this example, Saccharomyces cerevisiae (S. cerevisiae) KCCM12638 was used as the parent strain for UV random mutation.

### 1-5. Fed-Batch Culture of Yeast and Heme Production Concentration

Unlike batch culture, fed-batch culture can significantly increase the amount of microbial cells by continuously supplying carbon and nitrogen sources that are necessary for the growth of microorganisms, thereby increasing the amount of production of the desired substance. In the present invention, the heme production of Saccharomyces cerevisiae KCCM12638 strain was ultimately increased through fed-batch culture.

The fed-batch culture was performed in a 2.5 L bioreactor with a working volume of 1 L. The pre-culture was performed in the same manner as in Example 1-2. The pre-cultured yeast strain was inoculated into a baffled flask including 100 mL YP20D medium such that the optical density at 600 nm (OD600) became 1.0, and sub-culture was performed at 25° C. and 250 rpm for 48 hours. The sub-cultured yeast strain was inoculated into 1 L of 40YP20D ((4% (w/v) yeast extract (40 g/L), 2% (w/v) bacto peptone (20 g/L), 2% (w/v) glucose (20 g/L)) medium such that the optical density at 600 nm (OD600) became 1.0. The culture was performed at 25° C., 700 rpm, and 2 vvm with pH maintained at 3.5 or 4.5. When the initial glucose was completely consumed, additional feeding solution (feeding solution; 280 g/L yeast extract, 140 g/L bacto peptone, 350 g/L glucose) was injected at a flow rate of 4 to 24 mL/hr. The injection rate of the feeding solution was determined such that the glucose concentration of the yeast culture was close to 0 g/L and the ethanol concentration did not exceed 10 g/L. The dissolved oxygen (DO) was maintained at a high level by increasing the shaking speed and air injection speed from a minimum of 700 rpm and 2 vvm to a maximum of 1,200 rpm and 5 vvm, respectively. The concentrations of glucose, glycerol, acetate and ethanol in the medium were measured by high performance liquid chromatography (HPLC) (Thermo fisher Ultimate 3000) equipped with a Rezex ROA-organic acid H+ column (Phenomenex, Torrance, CA) and a refractive index (RI) detector. The column temperature was maintained at 70° C., and a 5 mM sulfuric acid (H2SO4) solution was flowed as a mobile phase at a flow rate of 0.6 mL/min for detection. The heme concentration was measured by the method described in Examples 1-4.

As a result of performing fed-batch culture in the same manner as above under pH 3.5 and pH 4.5 conditions, as confirmed in FIG. 2, a maximum of 37.3 mg/L heme was produced under pH 3.5 conditions, and as confirmed in FIG. 3, a maximum of 48.5 mg/L heme was produced under pH 4.5 conditions, confirming that pH 4.5 conditions are desirable for producing heme.

### EXAMPLE 2

### Production of Enhanced Strains Through Random Mutation

### 2-1. UV Random Mutation Production

50 μL of the yeast strain KCCM12638 glycerol stock stored in a deep freezer was inoculated into test tubes including 5 mL YP20D (1% (w/v) yeast extract (10 g/L), 2% (w/v) bacto peptone (20 g/L), 2% (w/v) glucose (20 g/L)) medium and pre-cultured at 25° C. and 250 rpm for 48 hours.

The pre-cultured yeast strain was diluted such that the optical density at 600 nm (OD600) was 1.0, and 0.2 mL thereof was collected by centrifugation. The recovered microorganisms were released into YP20D and then transferred to a PCR tube. In order to produce UV random mutations, PCR tubes containing yeast cells were placed under a 254 nm UV lamp at a distance of 30 cm at an irradiation dose of 3,000 to 9,000 mW/cm² for 2 to 12 hours. As shown in FIG. 4, it was confirmed that the death rate was 97.8% at 2 hours after UV irradiation, and this time period was selected as the optimal UV irradiation time. The yeast cells that were UV irradiated for 2 hours were diluted 10³, 10⁴ and 10⁵ folds, and then, 0.1 mL was spread on YPD agar medium and cultured at 30° C. for 48 hours.

### 2-2. Confirmation of Heme Content of Mutant Strain

After UV treatment, colonies that grew up were randomly selected and pre-culture and main culture (baffle flask shaking culture) were performed as in Example 1 to analyze the heme concentration. Among the analyzed colonies, strains with higher heme content than the parent strain KCCM12638 were selected. The culture performance (OD600) and heme content according to the results are as shown in Table 3 below.

TABLE 3

48 Hours

| Strain | OD600 | Heme (mg/L) |
|---|---|---|
| KCCM12638 | 22.8 | 3.78 |
| KCTC 15340BP (HS-Y007) | 24.36 | 4.57 |

As can be seen from the results in Table 3, the OD600 of the tested strains was about 7% higher than that of the parent strain, and the heme content also increased by 21%. The strain with the heme content increased by about 1.21 times was named Saccharomyces cerevisiae HS-Y0071, and this strain was deposited at the Korea Research Institute of Bioscience and Biotechnology Korean Collection of Type Cultures (KCTC) on Mar. 7, 2023, and it was assigned Accession No. KCTC 15340BP.

### EXAMPLE 3

### Identification and SNP Analysis of UV Mutant Strains

The inventors of the present invention commissioned whole genome re-sequencing to Macrogen, and used Saccharomyces cerevisiae S288C as the whole genome to obtain SNP information between the parent strain KCCM12638 and the UV mutant strain HS-Y007 (Accession No. KCTC 15340BP). Whole genome resequencing is a method of analyzing mutations between specific individuals in a species with a whole genome (reference genome) and comparing the base sequences of individuals or populations. Whole genome resequencing can be used to confirm the distribution and pattern of mutations at the genome level.

First of all, the ITS1, 5.8S rRNA and ITS2 base sequences (hereinafter, referred to as the ITS portion) were extracted from the base sequences obtained through whole-genome resequencing, and it was sought to confirm whether HS-Y007 (Accession No. KCTC 15340BP) was the same Saccharomyces cerevisiae as the parent strain KCCM12638.

For comparison of the ITS portion, BLAST (https://blast.ncbi.nlm.nih.gov/) was performed to compare the ITS portion of S288C of the whole genome of the whole genome resequencing with that of Saccharomyces cerevisiae KCCM12638, which is the parent strain of the mutant. As confirmed in the comparison results in FIG. 5, 841 base sequences out of 848 KCCM12638 ITS portions were identical to S288C.

Subsequently, the ITS portions of the parent strain Saccharomyces cerevisiae KCCM12638 and the UV mutant strain Saccharomyces cerevisiae HS-Y007 (Accession No. KCTC 15340BP) were compared. As shown in FIG. 6, 848 out of 848 base sequences of the ITS portions of the Saccharomyces cerevisiae KCCM12638 strain and the Saccharomyces cerevisiae HS-Y007 (Accession No. KCTC 15340BP) strain were identical, confirming that the Saccharomyces cerevisiae HS-Y007 (Accession No. KCTC 15340BP) is also a Saccharomyces cerevisiae strain derived from KCCM12638.

Next, in order to determine whether the Saccharomyces cerevisiae HS-Y007 (Accession No. KCTC 15340BP) was an independently produced strain that was modified from KCCM12638, SNPs among the results of full-length genome resequencing analysis were used. Based on the chromosomal sequence of S288C, the results of comparing the SNPs of the KCCM12638 and Saccharomyces cerevisiae HS-Y007 (Accession No. KCTC 15340BP) are shown in Table 4. The chromosome sequence information of S288C, which serves as the standard, is disclosed in NCBI, and the NCBI ID information for each chromosome in Table 4 is shown in Table 5.

TABLE 4

| | | | | HS-Y007 |
|---|---|---|---|---|
| Chromosome | Position | S288C | KCCM12638 | (KCTC 15340BP) |
| 3 | 413 | C | A | T |
| 3 | 468 | G | T | A |
| 5 | 570,166 | A | G | T |
| 7 | 8,579 | A | T | C |
| 7 | 158,964 | C | T | A |
| 7 | 171,975 | A | G | C |
| 8 | 364,680 | C | A | T |
| 10 | 502,139 | G | A | C |
| 12 | 1,064,659 | A | G | C |
| 12 | 1,064,798 | A | T | G |
| 12 | 1,064,817 | A | G | T |
| 14 | 783,791 | G | C | T |
| 15 | 30,149 | G | A | T |

TABLE 5

| Chromosome | Genbank ID | RefSeq ID |
|---|---|---|
| 3 | BK006937.2 | NC_001135.5 |
| 5 | BK006939.2 | NC_001137.3 |
| 7 | BK006941.2 | NC_001139.9 |
| 8 | BK006934.2 | NC_001140.6 |
| 10 | BK006943.2 | NC_001142.9 |
| 12 | BK006945.2 | NC_001144.5 |
| 14 | BK006947.3 | NC_001146.8 |
| 15 | BK006948.2 | NC_001147.6 |

As confirmed in Table 4, the Saccharomyces cerevisiae HS-Y007 (Accession No. KCTC 15340BP) has at least 13 SNPs compared to the KCCM12638 strain. Specifically, the Saccharomyces cerevisiae HS-Y007 (Accession No. KCTC 15340BP) has the SNPs of 413A>T and 468T>A mutations in chromosome 3, a 570166G>T mutation in chromosome 5, 8579T>C, 158964T>A and 171975G>C mutations in chromosome 7, a 364680A>T mutation in chromosome 8, a 502139A>C mutation in chromosome 10, 1064659G>C, 1064798T>G and 1064817G>T mutations in chromosome 12, a 783791C>T mutation in chromosome 14, and a 30149A>T mutation in chromosome 15.

As the specific parts of the present invention have been described in detail above, it is clear to those skilled in the art that these specific techniques are merely preferred embodiments and do not limit the scope of the present invention. Accordingly, the actual scope of the present invention will be defined by the appended claims and their equivalents.

### [Accession Number]

Name of Depository Institution: Korea Research Institute of Bioscience and Biotechnology Korean Collection of Type Cultures (KCTC)

Accession No.: KCTC15340BP

Date of Accession: Mar. 7, 2023

1.-18. (canceled)

19. An HS-Y007 mutant strain of Saccharomyces cerevisiae with enhanced heme productivity compared to a wild type, wherein the mutant strain is deposited under Accession No. KCTC 15340BP.

20. The HS-Y007 mutant strain of claim 19, wherein the HS-Y007 mutant strain is derived from Saccharomyces cerevisiae KCCM12638.

21. The HS-Y007 mutant strain of claim 19, wherein the HS-Y007 mutant strain is produced by irradiating Saccharomyces cerevisiae KCCM12638 with ultraviolet rays having a wavelength of 250 to 260 nm at a distance of 20 to 50 cm in the vertical direction at an irradiation dose of 3,000 to 9,000 mW/cm² for 1 to 2 hours.

22. The HS-Y007 mutant strain of claim 19, wherein the HS-Y007 mutant strain has single nucleotide sequence mutations of 413A>T and 468T>A mutations in chromosome 3, a 570166G>T mutation in chromosome 5, 8579T>C, 158964T>A and 171975G>C mutations in chromosome 7, a 364680A>T mutation in chromosome 8, a 502139A>C mutation in chromosome 10, 1064659G>C, 1064798T>G and 1064817G>T mutations in chromosome 12, a 783791C>T mutation in chromosome 14, and a 30149A>T mutation in chromosome 15, compared to Saccharomyces cerevisiae KCCM12638.

23. A method for producing the HS-Y007 mutant strain of claim 19, comprising:
a) pre-culturing Saccharomyces cerevisiae KCCM12638; and
b) irradiating the pre-cultured Saccharomyces cerevisiae KCCM12638 with ultraviolet rays.

24. The method of claim 23, further comprising:
a′) diluting the pre-cultured strain such that the optical density at 600 nm is 0.5 to 1.5, between step a) and step b).

25. The method of claim 23, wherein step b) is performed by irradiating Saccharomyces cerevisiae KCCM12638 with ultraviolet rays having a wavelength of 250 to 260 nm at a distance of 20 to 50 cm in the vertical direction at an irradiation dose of 3,000 to 9,000 mW/cm² for 1 to 2 hours.

26. The method of claim 23, further comprising:
c) selecting a mutant strain that has single nucleotide sequence mutations of 413A>T and 468T>A mutations in chromosome 3, a 570166G>T mutation in chromosome 5, 8579T>C, 158964T>A and 171975G>C mutations in chromosome 7, a 364680A>T mutation in chromosome 8, a 502139A>C mutation in chromosome 10, 1064659G>C, 1064798T>G and 1064817G>T mutations in chromosome 12, a 783791C>T mutation in chromosome 14, and a 30149A>T mutation in chromosome 15, compared to Saccharomyces cerevisiae KCCM12638, after step b).

27. A method for producing heme using the HS-Y007 mutant strain of Saccharomyces cerevisiae (Accession No. KCTC 15340BP) according to claim 19.

28. The method of claim 27, wherein the method comprises:
a) culturing an HS-Y007 mutant strain of Saccharomyces cerevisiae (Accession No. KCTC 15340BP); and
b) recovering heme from the HS-Y007 mutant strain.

29. The method of claim 28, wherein the HS-Y007 mutant strain of step a) is cultured in a medium comprising at least one selected from the group consisting of glucose, fructose or galactose as a carbon source, and yeast extract, peptone, tryptone, malt extract, beef extract, casein, soytone, sodium nitrate (NaNO₃), ammonium chloride (NH₄Cl) and ammonium sulphate ((NH₄)₂SO₄) as a nitrogen source.

30. The method of claim 29, wherein the carbon source is 3 to 7% (w/v) of glucose, fructose or galactose.

31. The method of claim 29, wherein the nitrogen source is 2 to 6% (w/v) of yeast extract and 0.1 to 3% (w/v) of peptone.

32. The method of claim 28, wherein the HS-Y007 mutant strain of step a) is cultured in a fed-batch manner at a pH of 3 to 6, with a shaking speed of 100 to 1,500 rpm, and air injection at a speed of 0.5 to 10 vvm.

33. A medium composition for culturing the HS-Y007 mutant strain of claim 19, comprising:
i) 3 to 7% (w/v) of glucose, fructose or galactose as a carbon source; and
ii) 2 to 6% (w/v) of yeast extract and 0.1 to 3% (w/v) of peptone as a nitrogen source.

34. The medium composition of claim 33, wherein the medium composition is a medium composition for producing heme.

35. A food composition, comprising the HS-Y007 mutant strain of claim 19, a culture product of the HS-Y007 mutant strain, heme produced from the HS-Y007 mutant strain or heme isolated from a culture product of the HS-Y007 mutant strain.

36. The food composition of claim 35, wherein the food is artificial meat or meat substitute food.
